(19) United States
(12) Patent Application Publication
(10) Pub. No.: US 2026/0345317 A1
(43) Pub. Date: Nov. 5, 2026

(54) STERILE MOIST PACKAGE FOR HYDROPHOBIC INTRAOCULAR LENS AND METHOD OF PACKING SAME

(71) Applicant: HANITA LENSES LTD., Kibbutz Hanita (IL)

(72) Inventors: Yakir Kushlin, Atlit (IL); Boris Hash, Maalot Tarshiha (IL); Alexander MALIAROV, Tirat Carmel (IL); Eran IVANIR, Kiryat Bialik (IL)

(21) Appl. No.: 19/104,995

(22) PCT Filed: Aug. 22, 2023
(86) PCT No.: PCT/IL2023/050890

§ 371 Date: Feb. 19, 2025

Related U.S. Application Data
(60) Provisional application No. 63/406,843, filed on Sep. 15, 2022.

(30) Foreign Application Priority Data
Aug. 23, 2022 (IL) 295879

(51) Int. Cl. A61F 2/16; A61L 2/07; A61L 2/206; A61L 103/20

(52) U.S. Cl. CPC A61F 2/1691; A61L 2/07; A61L 2/206; A61L 2103/20

(57) ABSTRACT

A sterile, moist package and packaging method for an intraocular lens (IOL). In some embodiments, the method comprises conducting steam sterilization inside an outer bag, with an IOL inside an open inner bag. The inner bag comprises a material that has a thermal conductivity >1.1 times the material of the IOL. The method further comprises sealing the inner bag via the outer bag, trapping humidified air (>70% RH at 25 °C) inside the inner bag. In other embodiments, the method comprises conducting ethylene oxide sterilization and humidification inside a first-sealed region of a sealable bag including a gas-permeable window—containing an IOL preloaded in an IOL injector. The method further comprises sealing the sealable bag with a second seal, such that a second-sealed region of the sealable bag excludes the gas-permeable window, trapping humidified air (>60% RH at 25 °C) inside the second-sealed region of the sealable bag.

### RELATED APPLICATIONS

The present application claims the priority benefit of Israel application 295879 filed on 23 Aug. 2022 and U.S. provisional application 63/406,843 filed on 15 Sep. 2022. Both priority applications are incorporated herein by reference.

### FIELD OF THE INVENTION

The present invention relates generally to a sterile package and method of packaging suitable for intraocular lenses. More specifically, the present invention relates to a sterile package filled with moisture for intraocular lens and method of packing same.

### BACKGROUND OF THE INVENTION

An intraocular lens (IOL) is an artificial lens that replaces the eye's natural lens which is removed during cataract surgery or refractive lens exchange (RLE) surgery. IOLs usually comprise a polymer-based lens with polymer-based side struts, or extensions, called haptics, to hold the lens in place in the capsular bag inside the eye. In most cases, the haptics are of the same material as the lens and the IOLs are called one-piece IOLs. IOLs' optical zones can be of different designs: monofocal, multifocal-refractive, multifocal-diffractive, with or without a toric correction element (used to correct preexisting corneal astigmatism prior to the surgery). IOL designs may be of constant mechanics or may include an accommodating mechanism, designed to allow the eye to regain some partial focusing ability in order to change focus from distance to near vision by using the ciliary muscle movement or other external energy source.

In general, modern soft or flexible IOLs are made from various polymers, such as those based an acrylate backbone, polypropylene-based with various side chains made of hydrophilic nature such as Hydroxy-Methyl-Meth-Acrylate or hydrophobic MethylMethacrylate, aromatic side chains or other waxy side chains such as Ethyl-Ethoxy-Meth-Acrylate, and also silicone-and siloxane-based lenses or other combinations of polymers.

Modern IOLs are made of a soft material enabling it to be injected into the eye through a small incision. Known polymeric materials used in ophthalmology have a certain amount of water content ranging from close to 0% of water content considered as the extreme hydrophobic up to around 40% as the extreme hydrophilic.

IOLs are sterilized in their primary packaging and can be sterilized in different forms, either by steam sterilization, ethylene oxide (EtO), gamma irradiation and various plasma sterilization methods. Hydrophilic IOLs have high water content and are usually supplied in a water or saline solution, the water content supporting them to maintain their flexibility. Thus, hydrophilic lenses are supplied in water/saline while hydrophobic lenses are mostly supplied in a dry packaging, mostly using EtO sterilization.

Soft IOLs are found to be more flexible once hydrated. Hydrophilic IOLs must be kept in water or saline in order to be implanted, as in the non-hydrated state the lens is rigid and not flexible. Soft IOLs made of a hydrophobic material, when kept in a sterile dry environment, are less flexible than in their hydrated state. Such lack of flexibility of the hydrophobic IOL impairs the smoothness of injection of the IOL into the lens capsule.

Accordingly, there is a need for a new form of packaging IOLs in a sterile and moist environment that will ensure that the IOL can be provided at optimal conditions for implanting.

### SUMMARY OF THE INVENTION

Some aspects of the invention are directed to a package for an item such as an intraocular lens (IOL), comprising: an inner sealed sterile bag comprising the IOL and air having at least 75% relative humidity (RH) at 25 °C, and an outer sealed sterile bag encompassing the inner sealed sterile bag, wherein at least one edge of the inner sealed sterile bag is sealed via at least one surface of the outer sealed sterile bag. In some embodiments, the air in the inner sealed bag is at least 80% RH.

In some embodiments, the inner sealed sterile bag comprises a material having a thermal conductivity higher by at least 1.1 times than the thermal conductivity of the IOL material. In some embodiments, the inner sealed sterile bag is made from at least one of: aluminum foil and polyester laminate with an aluminum layer. In some embodiments, the IOL inside the inner sealed sterile bag is glistening-free.

In some embodiments, the outer sealed sterile bag is a steam sterilization pouch. In some embodiments, the outer sealed sterile bag comprises a gas-permeable window such as a web of paper or polyethylene fibers. In some embodiments, the seal comprises polymeric adhesives suitable for steam sterilization. In some embodiments, the IOL is held by a holder inserted into the inner sealed sterile bag.

Some additional aspects of the invention are directed to a method for packing an intraocular lens (IOL), comprising: inserting the IOL into an inner sealable bag; inserting the inner sealable bag into an outer sealable bag; sealing the outer sealable bag; conducting steam sterilization of the inner sealable bag and inside the outer sealable bag; and sealing at least one opening of the inner sealable bag via at least one surface of the outer sealable bag.

In some embodiments, the method further comprises, prior to inserting the IOL into the inner sealable bag, placing the IOL on a holder, and inserting the holder into the inner sealable bag. In some embodiments, the inner sealed sterile bag has a thermal conductivity higher by at least 1.1 times than the thermal conductivity of the IOL material. In some embodiments, prior to the sealing, the method further includes inserting the portion of the holder further inside the inner sealable bag, such that surfaces of the at least one opening of the inner sealable bag are free to be attached and to be sealed to each other.

In some embodiments, sealing together the at least one opening of the inner sealable bag via the surface of the outer sealable bag comprises soldering the opening using polymeric adhesives. In some embodiments, the outer sealable bag further comprises a gas-permeable window.

Some additional aspects of the invention are directed to another method for packing an intraocular lens (IOL), comprising: inserting the IOL into an inner sealable bag; inserting the inner sealable bag into an outer sealable bag; sealing the outer sealable bag; conducting sterilization of the inner sealable bag and the outer sealable bag; inserting the sterilized packing into a humidity chamber; introducing humidity into the inner sealable bag, resulting in at least 75% relative humidity at 25 °C; and sealing at least one opening of the inner sealable bag via at least one surface of the outer sealable bag.

In some embodiments, the method further comprises, prior to inserting the IOL into the inner sealable bag, placing the IOL on a holder, and inserting the holder into the inner sealable bag. In some embodiments, the sterilization is done by EtO sterilization. In some embodiments, the outer sealable bag further comprises a gas-permeable window.

Some additional aspects of the invention are directed to a package for an item such as a hydrophobic intraocular lens (IOL), comprising: a bag containing the hydrophobic IOL, the bag being sterile and sealed, wherein the hydrophobic IOL has been sterilized by steam sterilization while inside the bag; and at least one of 1) the bag; 2) a holder of the IOL; and 3) a metallic object near the bag during the steam sterilization, comprises a material having a thermal conductivity higher by at least 1.1 times than the thermal conductivity of the hydrophobic IOL material; and the hydrophobic IOL in the sterile sealed bag is glistening-free. In some embodiments, the air in the bag has a relative humidity of at least 75% at 25 °C. In some embodiments, the air in the bag has a relative humidity of at least 80% at 25 °C.

In some embodiments, the sealed bag comprises two flexible layers bonded together along at least a portion of their circumference. In some embodiments, the two flexible layers are made of aluminum foil, polyester laminate with an aluminum layer, or any combination thereof.

Some additional embodiments are directed to a method for packaging a hydrophobic intraocular lens (IOL), comprising: inserting the hydrophobic IOL into a bag; conducting steam sterilization of the hydrophobic IOL inside the bag; and sealing the bag, wherein at least one of 1) the bag; 2) a holder of the hydrophobic IOL; and 3) a metallic object near the sealed bag during the steam sterilization, comprises a material having a thermal conductivity higher by at least 1.1 times than the thermal conductivity of the hydrophobic IOL material; and the hydrophobic IOL in the sterile, sealed bag is glistening-free.

In some embodiments, the method further comprises, prior to placing the IOL on the holder, connecting a stage to the holder. In some embodiments, the stage is made from a material having a thermal conductivity higher by at least 1.1 times than the thermal conductivity of the IOL material.

Some additional aspects of the invention are directed to a packaged intraocular lens (IOL), comprising: an IOL; an inner sealed, sterile bag comprising the IOL and air having at least 75% relative humidity at 25 °C; and an outer sealed, sterile bag encompassing the inner sealed sterile bag, wherein at least one edge of the inner sealed sterile bag is sealed via at least one surface of the outer sealed sterile bag.

Some additional aspects of the invention are directed to a packaged hydrophobic intraocular lens (IOL), comprising: a hydrophobic IOL; a bag containing the hydrophobic IOL, the bag being sterile and sealed, wherein at least one of 1) the bag; 2) a holder of the hydrophobic IOL; and 3) a metallic object near the bag during the steam sterilization, comprises a material having a thermal conductivity higher by at least 1.1 times than the thermal conductivity of the hydrophobic IOL material; the hydrophobic IOL, while inside the bag, was sterilized by steam sterilization, before the sealing of the bag; and the hydrophobic IOL in the sterile sealed bag is glistening-free.

Some additional aspects of the invention are directed to a method for packaging an IOL (or other item), comprising: placing the IOL inside a sealable bag, the sealable bag comprising an impermeable portion and a first gas-permeable window; sealing the sealable bag with a first seal, whereby the IOL and at least a portion of the first gas-permeable window are inside a first-sealed portion of the sealable bag; sterilizing the sealable bag in a sterilizing environment, the sterilizing environment permeating the first gas-permeable window and sterilizing the IOL; and sealing the sealable bag with a second seal, whereby the first gas-permeable window is outside the second-sealed portion of the sealable bag and the IOL is inside the second-sealed portion of the sealable bag, wherein the air inside the second-sealed portion is at least 60% relative humidity at 25 °C. In some embodiments, the air inside the second-sealed portion is at least 80% relative humidity at 25 °C.

In some embodiments, the IOL is composed of a hydrophobic material. In some embodiments, performing of sterilization is done by ethylene oxide (EtO) sterilization.

In some embodiments, the method further comprises placing the sealable bag in a humid environment, the humid environment thereby permeating the first gas-permeable window and humidifying the first-sealed portion to at least said relative humidity.

In some embodiments, the method further comprises holding of the IOL by an IOL holder. In some embodiments, the method further comprises loading the IOL into an IOL injector or IOL injector cartridge.

In some embodiments, the method further comprises placing the IOL holder, IOL injector, or IOL injector cartridge into an inner receptacle having a second gas-permeable window. In some embodiments, the inner receptacle comprises a blister pack with a gas-permeable backing. In some embodiments, the front part of the blister pack is impermeable.

In some embodiments, the method further comprises detaching a remnant from the sealable bag, the remnant being outside of the second-sealed portion.

In some embodiments, the sterile environment and the humid environment are provided by steam sterilization. In some embodiments, at least one of 1) impermeable portion of the sealable bag; 2) the holder; and 3) a metallic object near the holder has at least 1.1 times the thermal conductivity of the IOL.

In some embodiments, the method further comprises placing a humidity control pack in the sealable bag, thereby achieving, or regulating, said relative humidity in the second-sealed portion of the sealable bag.

Some aspects of the invention relate to a package for an IOL (or other item), comprising: a sealable bag, comprising an impermeable portion and a first gas-permeable window, the sealable bag configured for placing the IOL therewithin; the sealable bag is further configured to be sealed with a first seal, whereby the item and at least a portion of the first gas-permeable window are within a first-sealed portion of the sealable bag, wherein: the sealable bag is configured for being placed in a sterilizing environment, the sterilizing environment thereby permeating the first gas-permeable window and sterilizing the item; the sealable bag further comprises a second seal, wherein the first gas-permeable window is outside a second sealed portion of the sealable bag and the item is inside the second-sealed portion of the sealable bag; and the humidity in the second-sealed portion is at least 60% relative humidity at 25 °C.

In some embodiments, the IOL is composed of a hydrophobic material. In some embodiments, the sterilizing is done by ethylene oxide (EtO) sterilization.

In some embodiments, the sealable bag is further configured for being placed in a humid environment, the humid environment thereby permeating the first gas-permeable window and humidifying the first-sealed portion to at least said relative humidity.

In some embodiments, the IOL is held in an IOL holder. In some embodiments, the IOL is loaded inside of an IOL injector or IOL injector cartridge. In some embodiments, the package further comprises an inner receptacle containing the IOL holder, the IOL injector, or the IOL injector cartridge, inside the sealed bag, the inner receptacle having a second gas-permeable window. In some embodiments, the inner receptacle comprises a blister pack with a gas-permeable backing. In some embodiments, the front part of the blister pack is impermeable.

In some embodiments, a remnant of the sealable bag outside of the second sealed portion is detachable.

In some embodiments, the sterilization and humidification are done by steam sterilization. In some embodiments, one or more of the impermeable portion of the sealed bag and the holder has at least 1.1 times the thermal conductivity of the IOL.

In some embodiments, the package further comprises a humidity control pack in the second-sealed portion, thereby achieving, or regulating, the relative humidity.

Some aspects of the invention are directed to a packaged IOL, comprising: an IOL; and a sealable bag, comprising an impermeable portion and a first gas-permeable window, the sealable bag configured for placing the IOL therewithin; the sealable bag is further configured to be sealed with a first seal, whereby the IOL and at least a portion of the first gas-permeable window are within a first-sealed portion of the sealable bag, wherein the sealable bag is configured for being placed in a sterilizing environment, the sterilizing environment thereby entering the first gas-permeable window and sterilizing the IOL; and the sealable bag further comprises a second seal, wherein the first gas-permeable window is outside a second sealed portion of the sealable bag and the item is inside the second-sealed portion of the sealable bag; and the air inside the second-sealed portion is at least 60% relative humidity at 25 °C. In some embodiments, the relative humidity is at least 80% at 25 °C.

Some embodiments further comprise an IOL injector or IOL injector cartridge, into which the IOL is preloaded. In some embodiments, the IOL injector comprises a hydrophilic coating of its tip.

### BRIEF DESCRIPTION OF THE DRAWINGS

The invention, both as to organization and method of operation, together with objects, features, and advantages thereof, may best be understood by reference to the following detailed description when read with the accompanying drawings in which:

FIGS. 1A-B depict an intraocular lens (IOL) sterile, moist package according to some embodiments.

FIG. 1C depicts an IOL holder to be inserted into an inner bag according to some embodiments.

FIG. 2A is a flowchart of a method for packing an IOL, according to some embodiments.

FIG. 2B depicts an IOL package during several steps of the method of FIG. 2A, according to some embodiments.

FIG. 3 is a flowchart of another method of packing an IOL according to some embodiments of the invention.

FIGS. 4A-D depict a sterile, moist package for an item, at various stages of the packaging process, according to some embodiments.

FIGS. 5A-F depict a sterile, moist package for an IOL loaded in an IOL injector, at various stages of the packaging process, according to some embodiments.

FIG. 6 is a flowchart of a method for sterile, moist packaging of an item, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS

One skilled in the art will realize the invention may be embodied in other specific forms without departing from the spirit or essential characteristics thereof. The foregoing embodiments are therefore to be considered in all respects illustrative rather than limiting of the invention described herein.

### A. First Set of Embodiments

An advantage of steam sterilization is that it may provide both sterilization and hydration. However, sterilizing IOLs with steam will most likely cause development of micro-vacuoles inside the material of the optical part of the lens, also referred to in the industry as glistening, thus compromising the optical quality of the lens. Presently, the most commonly used method for sterilizing glistening-free hydrophobic IOLs is using ethylene oxide (EtO) which is a toxic gas that should be totally removed from the lens in the package prior to sealing the lens in a package after sterilization. The outcome of this process is a totally dry lens as any liquid sterilized by EtO will dissolve EtO residue in it forming ethylene glycol (thus making it toxic). Sterilization of a humid load is not viable by EtO, as well as by plasma, x-ray, gamma ray, or e-beam. Therefore, after these methods of sterilization, additional post-sterilization humidification of the lens and its storage environment may be required.

Some aspects of the invention are directed to a sterile, moist package for a hydrophobic IOL and a method of packing the IOL in order to ensure both a moist environment and sterile conditions. The IOL packed in the sterile, moist package may be glistening-free even after having being sterilized with steam. This may be achieved by selecting a material of an inner bag holding the IOL having a thermal conductivity higher by at least 1.1 times than the thermal conductivity of the IOL material. Such an IOL pack may keep the IOL sterile during the entire shelf life, and thus, no mold, fungus or any other moisture-related contaminations will be formed inside the sterile bag.

In some embodiments, the IOL package may include two bags, an inner bag holding the IOL inserted into an outer bag suitable for steam sterilization.

As used herein a “bag” refers to a package made from two flexible layers circumferentially bonded together along at least a portion of their circumference. A “bag” can be an envelope, a package, a parcel, a container, a wrap or any other type of encapsulation. At an “open” state the bag may have at least one opening in the bonded circumference allowing items (e.g., IOL or other bags) to be inserted into the “bag.”

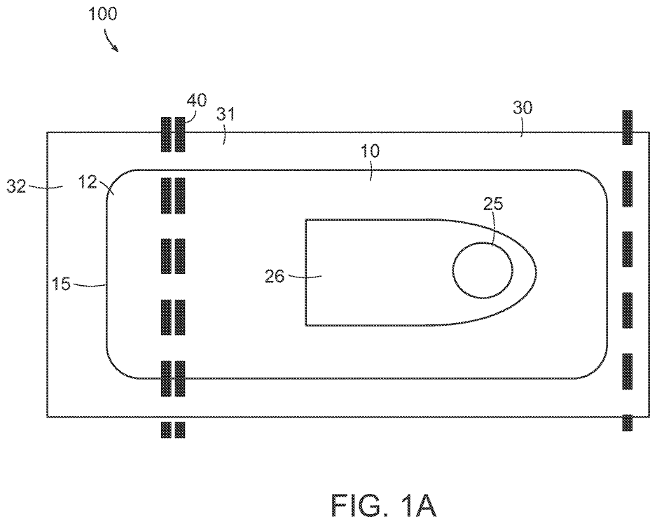

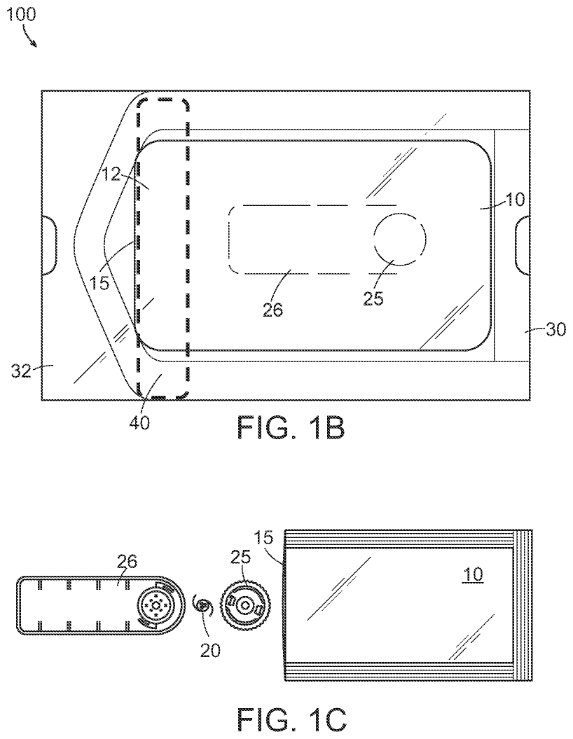

Reference is now made to FIGS. 1A and 1B, depicting an intraocular lens (IOL) sterile, moist package 100 according to some embodiments. An IOL package 100 may include an inner sealed sterile bag 10 comprising an IOL 20 and air having at least 75% relative humidity at 25 °C. In some embodiments, the air inside inner bag 10 may have at least 80% relative humidity at 25 °C, 85% relative humidity at 25 °C, 90% relative humidity at 25 °C, 95% relative humidity at 25 °C, 100% relative humidity at 25 °C, or any value in between. In some embodiments, a gas or gases besides air may be intermixed with or replace the air.

In some embodiments, inner sealed sterile bag 10 comprises a material having a thermal conductivity higher by at least 1.1 times than the thermal conductivity of the IOL material. In some embodiments, the material of inner sealed sterile bag 10 may have thermal conductivity index higher by at least 1.5, 1.8, 2, 2.5, 3, 4, 5, 10, 50, 100, 200, 500, 1000, 2000, 4000 and any value in between, times than the thermal conductivity index of the IOL material, under the same ambient conditions. For example, if the IOL is made from any type of hydrophobic acrylate the inner bag may be made from at least one of: aluminum foil, a polyester laminate with an aluminum layer and the like. In a non-limiting example, the thermal conductivity of the IOL is ~0.2 W/(m·K) while the thermal conductivity of the inner bag is made from aluminum foil is ~240 W/(m·K).

Such requirements for thermal conductivity of the inner sealed sterile bag 10 may not be applicable to EtO sterilization, in which there is no concern for condensation vaporization (unlike steam sterilization, as further described herein).

The inner sealed bag 10 can be rectangular, as illustrated, or can have any suitable shape allowing the insertion of IOL 20 and IOL holder 25 and stage 26 holding IOL 20, into bag 10, as shown in the image of FIG. 1C. In some embodiments, IOL holder 25 and stage 26 may also be made from a material having a thermal conductivity index higher by at least 1.1 times than the thermal conductivity index of the IOL material.

IOL package 100 may further include an outer sealed sterile bag 30 encompassing the inner sealed sterile bag 10. In some embodiments, at least one edge 12 of the inner sealed sterile bag 10 is sealed via at least one surface 31 of the outer sealed sterile bag 30 by seal 40. In some embodiments, the outer sealed sterile bag 30 may be a steam sterilization pouch. In some embodiments, the outer sealed sterile bag 30 may be an EtO sterilization pouch. In a non-limiting example, the outer sealed sterile bag 30 may comprise a gas-permeable window (not shown) such as a web of paper or polyethylene fibers, enabling penetration of humidity through the outer bag to reach the inner bag. For example, one (e.g., front) surface 31 of outer sterile bag 30 may comprise an impermeable layer, while the opposite (e.g., rear) surface may comprise a web of paper or polyethylene fibers, serving as a gas-permeable window.

In some embodiments, seal 40 may be formed by polymeric adhesives suitable for steam sterilization. In a non-limiting example, the seal may include polypropylene.

In some embodiments, IOL 20 is glistening-free despite being in a moist environment, for example, due to being sterilized by steam or by introducing humidity into inner sealed sterile bag 10 following dry sterilization methods.

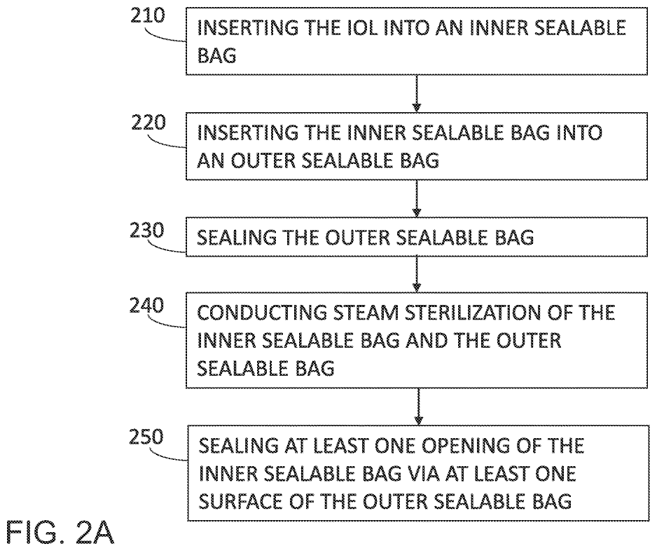

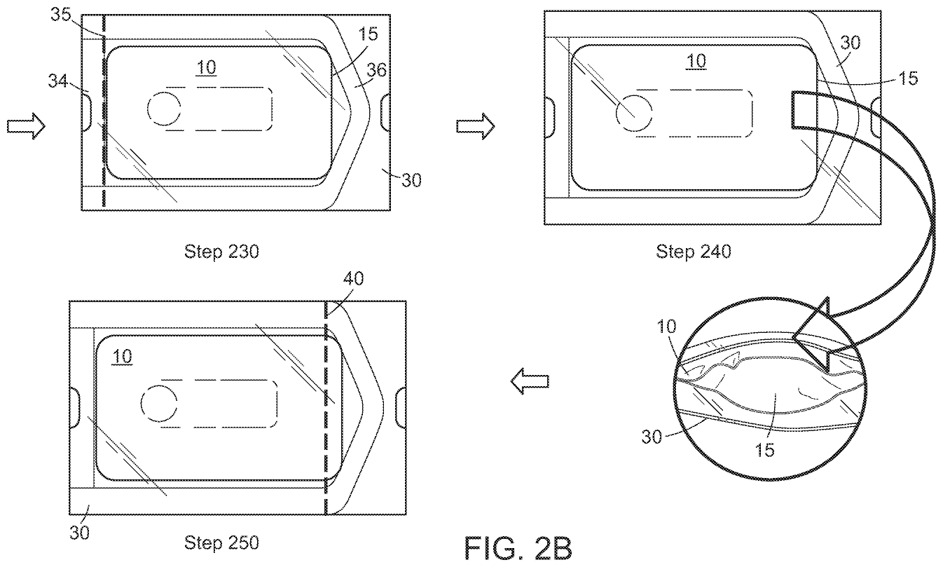

Reference is now made to FIGS. 2A and 2B, a flowchart and depictions of an IOL package, respectively, during steps in a method of packing an IOL according to some embodiments of the invention. In step 210, the IOL may be inserted into an inner sealable bag. In some embodiments, the inner sealable bag may have a thermal conductivity higher by at least 1.1 times than the thermal conductivity of the IOL material. In some embodiments, prior to inserting the IOL into the inner sealable bag, placing the IOL on a holder, and inserting the holder into the inner sealable bag. For example, as shown in FIG. 1C an IOL 20 may be placed on stage 26, which then may be connected to holder 25 to be inserted into inner bag 10 via at least one opening 15. In some embodiments, inner bag 10 may include two openings 15 from two opposite sides of inner bag 10.

In step 220, the inner sealable bag may be inserted into an outer sealable bag. For example, as shown in step 220 of FIG. 2B, inner sealable bag 10 may be inserted into outer sealable bag 30 via opening 34. In the non-limiting example, illustrated in FIG. 2B opening 15 of inner bag 10 is inserted first facing the bonded portion 36 of outer bag 30.

In step 230, the outer sealable bag may be sealed. For example, a first seal 35 may then be made to close the lip of outer bag 30. In some embodiments, seal 35 may include any adhesives suitable for steam sterilization.

In step 240, steam sterilization of the inner sealable bag and the outer sealable bag may be conducted, using any known method. For example, at least one opening section 15 in inner bag 10 (shown in FIG. 1A and FIG. 2B) may be used for introducing steam into inner bag 10.

As known in the art during steam sterilization, the vacuum cycle therein causes rapid evaporation of the condensate (or any other liquid water droplets) deposited on the coolest material. In order to evaporate the condensed droplets, an addition of vaporization calories (heat) may be required which it draws from the material, which therefore may rapidly cool. In some embodiments, placing the IOL in close proximity to a material of high thermal conductivity, such as, inner bag 10 and/or holder 25, during steam sterilization allows for avoiding water vapor pre-condensation on the IOL. In some embodiments, such an arrangement will prevent any condensation on the IOL, since the highly conductive material (e.g., bag 10 and/or holder 25) has a significantly larger surface area than the IOL surface area and may therefore cool before the IOL cools. The prevention of condensation on the IOL will prevent any form of glistening and thus maintain the optical quality of the IOL.

In some embodiments, in the case of heating of package 100, the heating may evaporate the water from the highly conductive material's surface instead of boiling the water on the IOLs surface.

A metallic object (or other object having a thermal conductivity higher by at least 1.1 times than the thermal conductivity of the IOL material) may be placed near the inner bag 10 during steam sterilization 240, alternatively or in addition to the inner bag and/or holder having a thermal conductivity higher by at least 1.1 times than the thermal conductivity of the IOL material. The metallic object may be inside or outside of the inner bag 10.

In step 250, at least one opening 15 of the inner sealable bag may be sealed via at least one surface of the outer sealable bag. For example, the opening 15 of the inner bag 10 is sealed via (through) the surface of the bag 30 by seal 40, for example, by soldering together all openings using polymeric adhesives. In some embodiments, if two openings 15 are included in inner bag 10, two seals 40 may be performed, one for sealing each opening. In some embodiments, prior to the sealing, the method further comprises inserting the portion of the holder 25 further inside the inner sealable bag 10, such that surfaces of the at least one opening 15 of the inner sealable bag 15 may be free to be attached and to be sealed to each other.

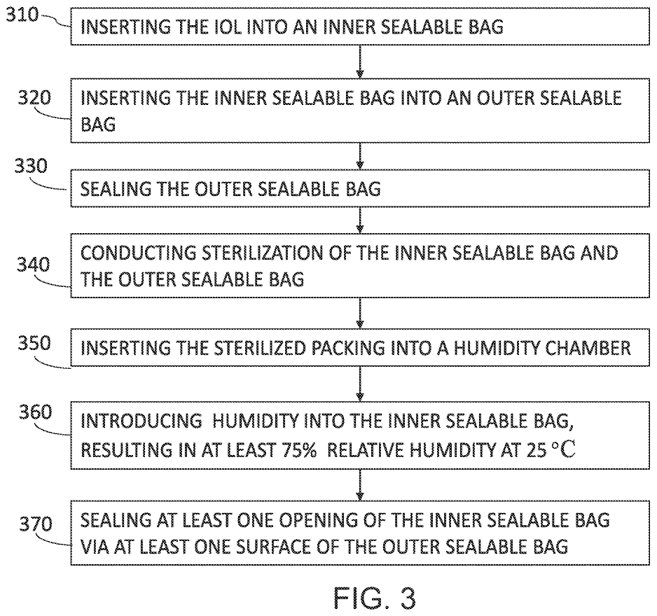

Reference is now made to FIG. 3, a flowchart of another method of packing an intraocular lens (IOL) according to some embodiments of the invention. In some embodiments, intraocular lens (IOL) package 100 may be packed using any one of the methods of FIG. 2A, FIG. 3 or using any suitable method enabling addition of humidity to a sterile sealed bag while maintaining the sterile conditions.

Steps 310, 320, and 330 of the method of FIG. 3 may be substantially similar to steps 210, 220 and 230 of the method of FIG. 2A. In step 340, sterilization of the inner sealable bag and the outer sealable bag may be conducted. For example, the sterilization may be conducted using the EtO method, in which EtO gas is provided during the sterilization process. EtO is a toxic gas, therefore must be totally removed prior to sealing the IOL in a package. Following the removal of the EtO, the inner bag 10 includes a totally dry IOL 20.

In step 350, the sterilized packing may be inserted into a humidity chamber. In step 360, humidity may be introduced into the inner sealable bag, resulting in at least 75% relative humidity at 25 °C.

In step 370, at least one opening of the inner sealable bag may be sealed via at least one surface of the outer sealable bag. For example, opening 15 of inner bag 10 is sealed via (through) the surface of the bag 30 by seal 40, for example, by soldering together all openings using polymeric adhesives. In some embodiments, prior to the sealing inserting the portion of the holder 25 further inside the inner sealable bag 10, such that surfaces of the at least one opening 15 of the inner sealable bag 15 may be free to be attached and to be sealed to each other.

### B. Second Set of Embodiments

Most existing IOL delivery systems (e.g., IOL injectors) and sometimes the IOL itself cannot withstand temperatures above 45 °C. In such cases, steam and dry heat sterilization are not viable. Most IOL delivery systems are also not compatible with radiation, plasma, and ozone sterilization. EtO sterilization may be a suitable sterilization process, but the most significant limitation of EtO sterilization is the inability to sterilize liquids, moist and humid objects, as the liquids dissolve the EtO residue into themselves.

Some embodiments relate to a hydrophobic intraocular lens packaging procedure for various IOL configurations, such as: a non-preloaded lens (an IOL, which may be mounted in an IOL holder), a fully preloaded lens (a lens in an IOL injector), and a semi-loaded lens (a lens loaded into an IOL injector cartridge, to be loaded into an IOL injector by the practitioner). The procedure may entail EtO sterilization (in some embodiments, steam sterilization) and packaging methods, which, when combined enable introducing liquid content into a package containing an IOL after EtO sterilization, thereby: 1) preventing drying the IOL-injector and its materials, e.g. coatings, which may result in unintended behavior during IOL injection, such as jams, discontinuous motion, and sudden ejections; and 2) maintaining high flexibility of the IOL and smooth translation during injection.

Some embodiments entail a package comprising a vapor-and gas-impermeable sterile barrier, suitable for EtO (and possibly steam) sterilization through a sealable vapor-and gas-permeable window (hereinafter, “gas-permeable window”). The package may be used for shipping and storage of a hydrophobic intraocular lens that is free of glistening (typically caused by steam sterilization). The packaged hydrophobic IOL may be sealed together with entrapped water vapor obtained during a humidification process which allows maintaining very high relative humidity level while the terminally sterile environment may be retained inside a sealed bag until the very moment of use during an ophthalmic surgical procedure. In some embodiments, the IOL configuration is further nested in a sterile barrier system, comprising an inner receptacle with a gas-permeable window, placed inside the sealed bag.

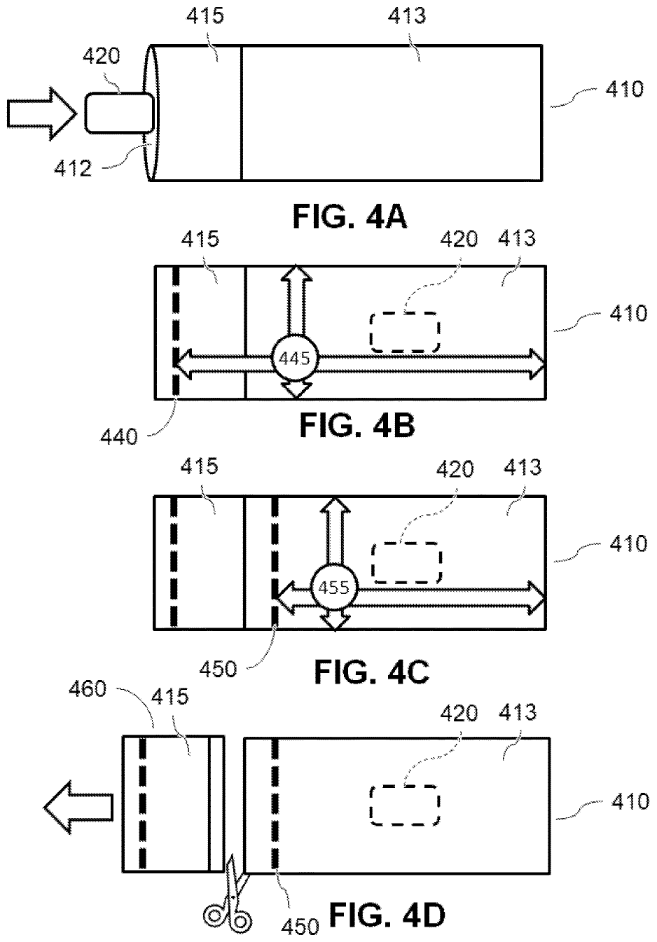

Reference is now made to FIG. 4A-4F, depicting a package for sterile, moist packaging of an item 420, such as an IOL, at various stages of the packaging process, according to some embodiments.

The package comprises a sealable bag 410. The sealable bag 410 comprises an impermeable portion 413 and at least one gas-permeable window 415 (hereinafter, first gas-permeable window 415). The first gas-permeable window 415 can be made of a material such as synthetic flashspun high-density polyethylene fibers, known commercially as Tyvek®. The sealable bag 410 can be an aluminum laminate pouch, known in the art as a header bag, with ~0 moisture vapor transmission rate (MVTR). The first gas-permeable window 415 may be on any portion of the sealable bag 410. In some embodiments of the package, the degree of gas permeability varies over the surface of the gas permeable opening 415.

The item 420 may be placed inside the sealable bag 410, as shown in FIG. 4A. Placement of the item 420 may be through an insertion opening 412 of the sealable bag 410. The insertion opening 412 may be at an edge of the sealable bag 410 or any other position of the sealable bag 410.

The sealable bag 410 may be sealed with at least one seal 440 (hereinafter, first seal 440). The item 420 and at least a portion of the first gas-permeable window 415 are inside a first-sealed portion 445 of the sealable bag 410 sealed by the first seal 440, as shown in FIG. 4B.

It is understood that the first seal 440 may comprise multiple seals (e.g., if there are multiple gas-permeable windows), as long as the first-sealed portion 445 is completely sealed by the first seal 440 and at least a portion of one first gas-permeable window 415 is within the first-sealed portion 445.

If the nature of the item 420 permits (e.g., size, shape, non-fragility of the item 420), the first seal 440 may be a complete seal around the edges of two separate layers of the sealable bag 410 while the item 420 is between the two layers.

The sealable bag 410 with the first seal 440 may be sterilized by placing the sealable bag 410 in a sterilizing embodiment. The first gas-permeable window allows the sterilizing environment to permeate into the first sealed-portion 445 of the sealable bag 410. The sterilizing environment sterilizes the inner surface area of the first-sealed portion 445 of the sealable bag 410 and the surfaces of the item 420. The item 420 can remain sterilized until the package is opened.

Sterilization may be, for example, by EtO sterilization or steam sterilization. EtO sterilization may be used, for example, when the item 420 cannot withstand the high temperature reached during steam sterilization.

Where steam sterilization is used for sterilizing an IOL, such as a hydrophobic IOL, the material of the sealable bag 410 (e.g., the impermeable portion 413) may have a thermal conductivity that is at least 1.1 times that of the material of the IOL. Alternatively, or additionally, the material of a holder (further described herein, including in reference to FIGS. 1A-C) in the sealable bag 410, holding the IOL, has a thermal conductivity that is at least 1.1 times that of the material of the IOL.

The interior of the sealable bag 410 may be humidified. Humidification may be achieved by placement of the sealable bag 410 in a humid environment, such as in a humidifier, incubator, or weather chamber. The humid environment thereby permeates the first gas-permeable window 415 and humidifies the interior of the sealable bag 410.

Where steam sterilization is used, the steam sterilization may serve both to sterilize and humidify the interior of the sealable bag 410.

Alternatively, or additionally, humidification may be achieved by a humidity controlling substance in the second sealed portion 455. For example, a humidity control pack, such as one supplied by Boveda, Inc., is inserted in the sealable bag 410 with the item 420. In some embodiments, an 84% RH humidity control pack is inserted in the sealable bag 410. In some embodiments, another method of humidification (i.e., steam sterilization, a humidifier) is initially used, and the humidity control pack is inserted to regulate the RH at a constant level.

The sealable bag 410 may be sealed with a second seal 450. The first gas-permeable window 415 may be outside a second-sealed portion 455 of the sealable bag 410 sealed by the second seal 450; the item 420 may be inside the second-sealed portion 455, as shown in FIG. 4C. The second seal 450 may prevent any gases or vapors from leaving or entering the second-sealed portion 455 of the sealable bag 410. Humidity may be thereby trapped in the second-sealed portion 455 until the package is opened. The air inside the second-sealed portion 455 may be at least 60% relative humidity (RH) at 25 °C. In some embodiments, the RH is at least 65% at 25 °C. In some embodiments, the RH is at least 80% at 25 °C. In some embodiments, the RH is at least 84% at 25 °C. In some embodiments, the package, upon sealing with the second seal 450 enables maintaining at least 90% of the initial humidity level inside the second-sealed portion 455 for five years after sealing with the second seal 450. In some embodiments, the gas and vapor in the second-sealed portion 455 is over-pressurized (e.g., by maintaining a cool environment during humidification and sealing the second seal 450), to prevent entry of the environment surrounding the package into the second-sealed portion 455.

Optionally, after the second seal 450 is made, a remnant 460 of the sealable bag 410 is detached from the rest of the bag 410 (e.g., by cutting), as shown in FIG. 4D. The remnant 460 is outside of the second-sealed portion 455, and preferably the remnant 460 does not include any portion of the second seal 450. Detachment of the remnant 460 reduces the amount of packaging material, facilitating storage and shipment of the package.

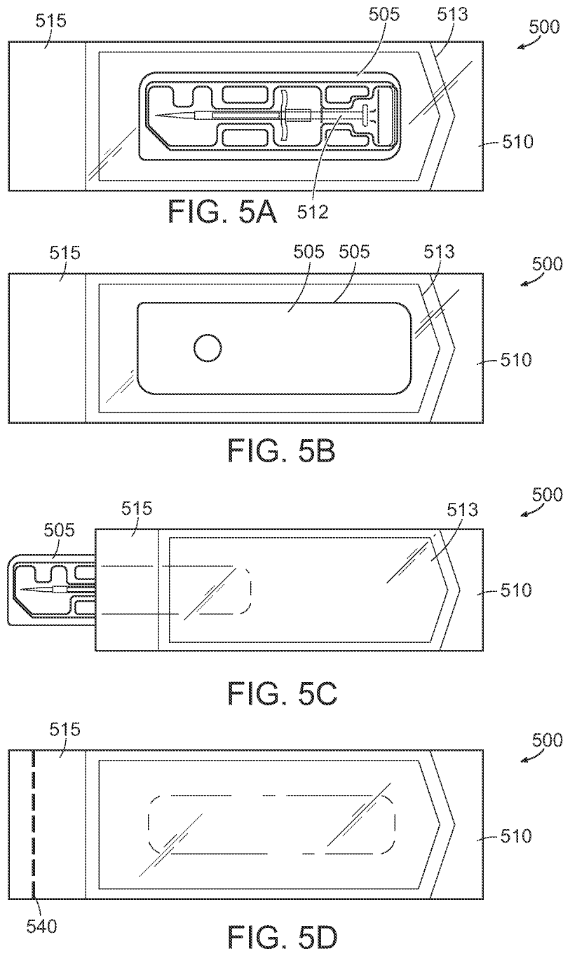

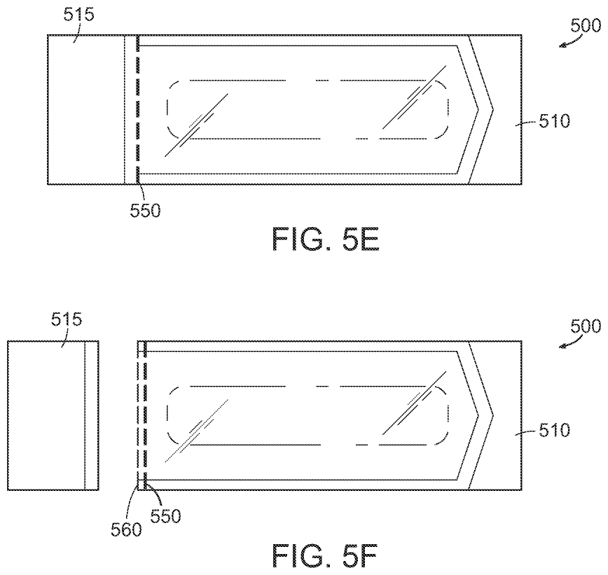

Reference is now made to FIGS. 5A-5F, depicting a sterile, moist package 500 for an IOL loaded in an IOL injector, at various stages of the packaging process, according to some embodiments.

The package comprises a sealable bag 510. The sealable bag 510 comprises an impermeable portion 513 and a gas-permeable window 515 (hereinafter, first gas-permeable window 515). The first gas-permeable window 515 can be made of a material such as synthetic flashspun high-density polyethylene fibers, known commercially as Tyvek®. The sealable bag 510 may be what is known in the art as a header pouch, commonly used for sterilization of medical devices.

The package 500 may comprise an inner receptacle 505. The inner receptacle 505 may comprise a second gas-permeable window 507. The inner receptacle can be 505 a blister pack, such as one made of polyethylene terephthalate glycol-modified (PET-G). The second gas-permeable window 507 can be a gas-permeable backing 507 of the blister.

The first gas-permeable window 515 may comprise a material such as synthetic flashspun high-density polyethylene fibers, known commercially as Tyvek®. In some embodiments, the inner receptacle 505, besides the second gas-permeable window 507, is made of an impervious material. In some embodiments, the inner receptacle 505 is made entirely of a gas-permeable material.

During packaging, an intraocular lens (IOL, not shown) may be placed in the inner receptacle 505. In some embodiments, the IOL is made of a hydrophobic material.

In some embodiments, the IOL is preloaded in an IOL injector 512, which may be inserted into the inner receptacle 505, as shown in FIG. 5A. In other embodiments, the IOL is preloaded in an IOL injector cartridge (not shown), which may be inserted into an inner receptacle 505. An injector cartridge is intended to be used, for example, by insertion into a reusable IOL injector.

In some embodiments, the preloaded IOL injector 512 or IOL injector cartridge is placed directly in the sealable bag 510 (i.e., without being in an inner receptacle).

In some embodiments, an IOL, mounted on an IOL holder, is placed directly into the sealable bag 510. In some embodiments, the holder is attached to a stage, as further described herein.

In some embodiments, the holder (and the stage, if present) are placed in an inner receptacle 505, such as blister pack (having an appropriately fitted shape and size), with a gas permeable backing 507.

During packaging, the sealable bag 510 may be sealed with a first seal 540, as shown in FIG. 5D. The inner receptacle 505 and at least a portion of the first gas-permeable window 515 are inside a first-sealed portion (depicted by 445 in FIG. 4B) of the sealable bag 510 sealed by the first seal 540.

The interior of the inner receptacle 505 may be accessible-through the second gas-permeable window 507 and first gas-permeable window 515-to the atmospheric environment outside the sealable bag 510.

The sealable bag 510 containing the inner receptacle 505 may be placed in a sterilization environment and then sterilization may be performed. The sterilization sterilizes the sealable bag 510 and contents therein. In particular, the following surfaces may be sterilized:
1. The inner surface area of the sealable bag 510.
2. The inner and outer surfaces of the inner receptacle 505.
3. The inner and outer surfaces area of the IOL injector 512.
4. The outer surface area of the IOL, on both sides of the IOL.

In some embodiments, the sterilization is by ethylene oxide (EtO) sterilization. In other embodiments, the sterilization is by steam sterilization. EtO sterilization may be preferred where the sealable bag contains an IOL injector 512, since many IOL injectors cannot withstand temperatures of steam sterilization. EtO sterilization may also be preferred where the IOL itself cannot withstand temperatures of steam sterilization.

After sterilization, the sealable bag 510 containing the inner receptacle 505 may be placed in a humidity chamber. In some embodiments, humidity is introduced into the humidity chamber until the air inside the first-sealed portion, including the interior of the inner receptacle 505, is at least 60% relative humidity at 25 °C. Humidification can be of particular benefit in the packaging of an IOL injector: keeping the hydrophilic coating of the injector tip moist can prevent the coating from cracking. In some embodiments, relative humidity is at least 65% at 25 °C. In some embodiments, the relative humidity is at least 80% at 25 °C.

Note that a gas or gases besides air may be intermixed with or replace the air.

In some embodiments, after the humidification, the sealable bag 510 is sealed with a second seal 550, whereby the second gas-permeable 515 opening is outside a second-sealed portion (depicted by 455 in FIG. 4C) of the sealable bag 510 and the inner receptacle 505 is inside the second-sealed portion, as shown in FIG. 5E. The second seal 550 prevents any gases or vapors from leaving or entering the second-sealed portion of the sealable bag 510. Air with the humidity level established by humidification may thereby be trapped inside the second-sealed portion until the package is opened.

The second gas-permeable window 520 may be removed from the sealable bag 510 by cutting sealable bag 510 with a cut 560 outside of the second seal 550, as shown in FIG. 5F.

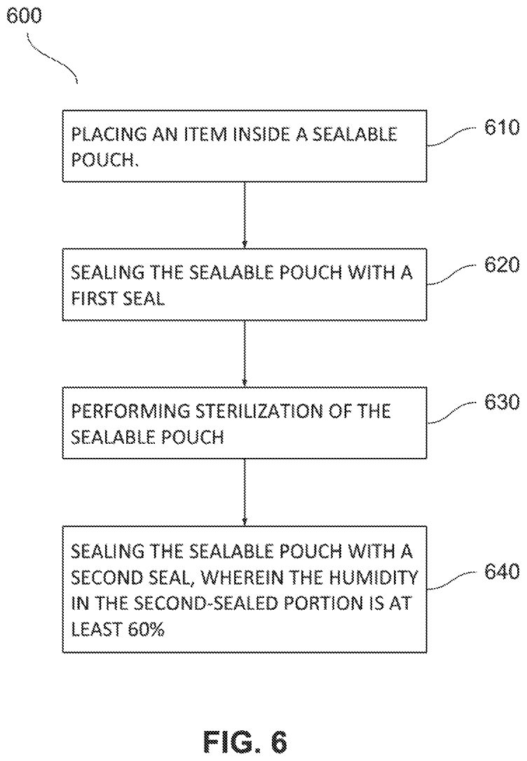

Reference is now made to FIG. 6, a flowchart of a method 600 for sterile, most packaging an item. The method 600 comprises:
1. Placing the item inside a sealable bag 610, the sealable bag comprising an impermeable portion and a first gas-permeable window.
2. Sealing the sealable bag with a first seal 620, whereby the item and at least a portion of the first gas-permeable window are inside a first-sealed portion of the sealable bag.
3. Sterilizing the sealable bag in a sterilizing environment 630, the sterilizing environment thereby permeating the first gas-permeable window and sterilizing the item.
4. Sealing the sealable bag with a second seal 640, whereby the first gas-permeable window is outside the second-sealed portion of the sealable bag and the item is inside the second-sealed portion of the sealable bag, and wherein the air inside the second-sealed portion may be at least 60% relative humidity at 25 °C.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may include features from other embodiments presented, including exchange of features between embodiments under the headings of “First Set” and “Second Set” in this Detailed Description. Embodiments not specifically described may include various features described herein.

While the description of packaging embodiments herein is in reference to packaging of an intraocular lens, it is understood that the packaging is suited to any medical device or other item requiring sterilization and/or benefiting from humid-environment storage.

1-29. (canceled)

30. A packaged intraocular lens (IOL), comprising:
an IOL;
an inner sealed sterile bag comprising the IOL and air having at least 75% relative humidity at 25 °C; and
an outer sealed sterile bag encompassing the inner sealed sterile bag, wherein:
the IOL inside the inner sealed sterile bag has been steam sterilized inside of the outer sealed sterile bag through at least one opening in the inner sealed sterile bag;
the inner sealed sterile bag comprises a material having a thermal conductivity at least 1.1 times the thermal conductivity of the IOL material; and
the steam sterilized IOL is glistening-free.

31. The packaged IOL of claim 30, wherein the IOL material is hydrophobic.

32. The packaged IOL of claim 30, wherein the inner sealed sterile bag is made from at least one of: aluminum foil and polyester laminate with an aluminum layer.

33. The packaged IOL according to claim 30, wherein said at least one opening of the inner sealed sterile bag has been sealed via at least one surface of the outer sealed sterile bag.

34. The packaged IOL according to claim 30, wherein the outer sealed sterile bag comprises a web of paper or polyethylene fibers.

35. A method for packaging an intraocular lens (IOL), comprising:
inserting the IOL into an inner sealable bag;
inserting the inner sealable bag into an outer sealable bag;
sealing the outer sealable bag; and
conducting steam sterilization of the inner sealable bag and inside the outer sealable bag through at least one opening in the inner sealable bag;
wherein:
the inner sealable bag comprises a material having a thermal conductivity at least 1.1 times the thermal conductivity of the IOL material; and
the steam sterilized IOL is glistening-free.

36. The method of claim 35, wherein the IOL material is hydrophobic.

37. The method of claims 35, further comprising, prior to inserting the IOL into the inner sealable bag, placing the IOL on a holder, and inserting the holder into the inner sealable bag.

38. The method of claim 35, further comprising sealing said at least one opening of the inner sealable bag via at least one surface of the outer sealable bag.

39. The method of claim 38, wherein prior to the sealing, the method further includes inserting the portion of the holder further inside the inner sealable bag, such that surfaces of the at least one opening of the inner sealable bag are free to be attached and to be sealed to each other.

40. A packaged intraocular lens (IOL), comprising
packaged contents comprising an IOL, a loaded IOL injector, or a loaded IOL injector cartridge;
an inner receptacle having a gas-permeable window, the contents being disposed therewithin, the inner receptacle having been sterilized in a sterilizing environment, the sterile environment thereby having permeated the gas-permeable window and sterilized the contents;
a sealable bag; and
a humidity control pack, wherein
the sterilized inner receptacle containing the contents and the humidity control pack are disposed and sealed within the sealable bag; and
the humidity control pack in the sealable bag regulates air inside the sealed bag to at least 60% relative humidity at 25 °C.

41. The packaged IOL of claim 40, wherein the air inside the sealed bag is at least 80% relative humidity at 25 °C.

42. The packaged IOL of claim 40, wherein said IOL is composed of a hydrophobic material.

43. The packaged IOL of claim 40, wherein the sterilizing is done by ethylene oxide (EtO) sterilization.

44. The packaged IOL of claim 40, wherein the inner receptacle comprises an impermeable blister pack with a gas-permeable backing.

45. The packaged IOL of claim 40, wherein the IOL injector comprises a tip with a hydrophilic coating.

46. A method for packaging an intraocular lens (IOL), comprising
placing contents to be packaged—comprising an IOL, a loaded IOL injector, or a loaded IOL injector cartridge—into an inner receptacle having a gas-permeable window;
sterilizing the inner receptacle in a sterilizing environment, the sterilizing environment thereby permeating the gas-permeable window and sterilizing the contents;
placing the inner receptacle inside a sealable bag;
placing a humidity control pack in the sealable bag; and
sealing the sealable bag,
wherein the humidity control pack in the sealable bag achieves or regulates air inside the sealed bag to at least 60% relative humidity at 25 °C.

47. The method of claim 46, wherein the air inside the sealed bag is at least 80% relative humidity at 25 °C.

48. The method of claim 46, wherein the IOL is composed of a hydrophobic material.

49. The method of claim 46, wherein the performing of sterilization is done by ethylene oxide (EtO) sterilization.
